SEQUENCE LISTING (1 sequence)

SEQ ID NO: 1 - DNA, 1359 bp, SORGHUM BICOLOR
atggtgctgcagcgcttctcgtggccgtacggcggccggagagcctccttctgcggcagcttcaccgggtggagggagtgtcccatggggctggtcggggccgagttccaggtcgtcttcgatctgcctcccggggtttaccagtaccggtttttggttgatggtgtctggaggtgtgatgaggcgaaaccctttgtacgtgatgaatatggattgatcagcaatgaagtgcttgtggaaaacaatgtacaacctgttgtgcagccagagccttctatcagaggaaccaatgtggatgagggtatcattttgacaacaatgcccccggagccatcatctcagaacccaggcgtgcaaatagcagttttccgccatgtggtctctgaaatattattacacaataccatatatgacgttgttcctatttctagcaagttagcagttttggacactcagcttcctgttaaacaagcatttaaaataatgcatgatgagggtcttgctctggttcctctttgggatgaccatcagggaactataacaggcatgctcactgcatcagattttgtattaatgttgagaaagttgcagagaaacattcgagttattggcaatgaagagcttgaaatgcatcccatttctgcttggaaagaagcaaagctacagttttatggtgggcctgatggtgctgccatgcagagaaggccattaatccatgttaaggattcagataatttagtggatgtggcattgactataatcagaaatgaaatatcttcagttcctatctttaagtgcgtgccagattcaacagggatgcctttccttagtcttgcaaccctccaggggattttgaaatttctttgctcgaagctacaagaacaggctgagggctgttcccttctgcacaatcagcttctcagtattcctattggcacatggtctccacatacgggaaggtcaagtagcaggcaactcagaactttgctactgagttctcctctaaatacctgcctggattttctgcttcaagatagagtaagctcaattcctatagttgatgacaatggatccctccgtgacgtctactcactcagtgatatcatggctctggcgaagaatgatgtttatgctcgcatcgaacttgaacaagtgactgtgcaaaatgctttggatgtgcaataccaggtgcatggccgaagacagtgtcatacttgtttacagacgagtaccttgctggaagtcttggagggattgtctgttccaggagtgcgacggcttgttgttattgaacaaagtaccagatttgtggaaggaatcatctcattgagagacatttttacatttctccttggatag

(19) United States
(12) Patent Application Publication
(10) Pub. No.: US 2021/0371871 A1
(43) Pub. Date: Dec. 2, 2021

(54) Pleiotropic Gene that Increases Biomass and Sugar Yield in Sorghum and Sugarcane

(71) Applicant: University of Louisiana at Lafayette, Lafayette, LA (US)

(72) Inventors: Yi-Hong Wang, Lafayette, LA (US); Jieqin Li, Fengyang (CN); Hari Upadhyaya, Andhra Pradesh (IN); Chudamani Sharma Prakash, Lafayette, LA (US); Lihua Wang, Fengyang (CN); Yanlong Liu, Fengyang (CN); Li Gao, Fengyang (CN); K Seetharam, Andhra Pradesh (IN); CLL Gowda, Andhra Pradesh (IN); K Ganesamurthy, Andhra Pradesh (IN); Shailesh Kumar Singh, Andhra Pradesh (IN)

(21) Appl. No.: 17/336,525

(22) Filed: Jun. 2, 2021

Related U.S. Application Data
(60) Provisional application No. 63/033,269, filed on Jun. 2, 2020.

(51) Int. Cl. C12N 15/82

(52) U.S. Cl. CPC C12N 15/8245; C12N 15/8261

(57) ABSTRACT

A method for increasing biomass and sugar yield of a plant comprising: transforming a plant with a first gene that is functional in a plant, wherein said gene is pleitropic, and wherein said plant overexpresses said gene, thereby increasing biomass and sugar yield; producing said plant through molecular breeding using information of said gene; and cultivating said plant.

### CROSS REFERENCE TO RELATED APPLICATIONS

This application claims priority to and the benefit of U.S. Provisional Patent Application No. 63/033,269 filed on Jun. 2, 2020 entitled “A Pleiotropic Gene that Increases Biomass and Sugar Yield in Sorghum and Sugarcane” and U.S. Provisional Patent Application No. 39/614,686 filed on Jun. 3, 2020 entitled “A Pleiotropic Gene that Increases Biomass and Sugar Yield in Sorghum and Sugarcane.”

### STATEMENT REGARDING FEDERALLY SPONSORED RESEARCH OR DEVELOPMENT

Not Applicable.

### REFERENCE TO A “SEQUENCE LISTING,” A TABLE, OR A COMPUTER PROGRAM

The Sequence Listing associated with this application is provided in text format in lieu of a paper copy, and is hereby incorporated by reference into the specification. The name of the text file is “SequenceListingWang.txt”, the date of creation is Aug. 3, 2021, and the size of the text file in bytes is 2.37 KB.

### DESCRIPTION OF THE DRAWINGS

The drawings constitute a part of this specification and include exemplary embodiments of the A Pleiotropic Gene that Increases Biomass and Sugar Yield in Sorghum and Sugarcane, which may be embodied in various forms. It is to be understood that in some instances, various aspects of the invention may be shown exaggerated or enlarged to facilitate an understanding of the invention. Therefore the drawings may not be to scale.

FIG. 1A is a quantitative trait locus (annotated arrow) on chromosome 6 associated with plant height, flowering time, fresh biomass, juice yield and Brix in sorghum, Manhattan plot for plant height in the Reference Set panel in the Mini Core panel.

FIG. 1B is a quantitative trait locus (annotated arrow) on chromosome 6 associated with plant height, flowering time, fresh biomass, juice yield and Brix in sorghum, Manhattan plot for flowering time in the Mini Core panel.

FIG. 1C is a quantitative trait locus (annotated arrow) on chromosome 6 associated with plant height, flowering time, fresh biomass, juice yield and Brix in sorghum, Manhattan plot for fresh biomass in the Mini Core panel.

FIG. 1D is a quantitative trait locus (annotated arrow) on chromosome 6 associated with plant height, flowering time, fresh biomass, juice yield and Brix in sorghum, Manhattan plot for juice yield in the Mini Core panel.

FIG. 1E is a quantitative trait locus (annotated arrow) on chromosome 6 associated with plant height, flowering time, fresh biomass, juice yield and Brix in sorghum, Manhattan plot for Brix in the Mini Core panel.

FIG. 2A is a Manhattan plot showing the pleiotropic locus in FIG. 1 mapped to average number of basal tillers in the Mini Core (MC) panel. X-axis represents chromosome physical distance in bp and Y-axis is −log(p), a measure of significance.

FIG. 2B is a Manhattan plot showing the pleiotropic locus in FIG. 1 mapped to average 100 seed weight in the Mini Core (MC) panel. X-axis represents chromosome physical distance in bp and Y-axis is −log(p), a measure of significance.

FIG. 3A is a quantitative trait locus on chromosome 6 associated with plant height, flowering time, fresh biomass, juice yield and Brix in sorghum, with genomic region associated with plant height. Location of the genes was based on Sbi v1.4.

FIG. 3B is a quantitative trait locus on chromosome 6 associated with plant height, flowering time, fresh biomass, juice yield and Brix in sorghum, with genomic region associated with flowering time.

FIG. 3C is a quantitative trait locus on chromosome 6 associated with plant height, flowering time, fresh biomass, juice yield and Brix in sorghum, with genomic region associated with fresh biomass.

FIG. 3D is a quantitative trait locus on chromosome 6 associated with plant height, flowering time, fresh biomass, juice yield and Brix in sorghum, with genomic region associated with juice yield.

FIG. 3E is a quantitative trait locus on chromosome 6 associated with plant height, flowering time, fresh biomass, juice yield and Brix in sorghum, with genomic region associated with Brix. Location of the genes was based on Sorghum bicolor v3.1.1.

FIG. 4A is a Box plot of Genes 4, 6 and 7 overexpression plants compared to control in biomass. X inside each box represents the mean and horizontal line the median value of each data group. ** indicates significant difference from control at p<0.01 level

FIG. 4B is a Box plot of Genes 4, 6 and 7 overexpression plants compared to control in plant height. X inside each box represents the mean and horizontal line the median value of each data group. ** indicates significant difference from control at p<0.01 level

FIG. 4C is a Box plot of Genes 4, 6 and 7 overexpression plants compared to control in Brix. X inside each box represents the mean and horizontal line the median value of each data group. ** indicates significant difference from control at p<0.01 level

FIG. 4D is a Box plot of Genes 4, 6 and 7 overexpression plants compared to control in tiller number. X inside each box represents the mean and horizontal line the median value of each data group. ** indicates significant difference from control at p<0.01 level.

FIG. 5 shows phenotypic difference of transgenic sorghum (42-1, 42-2) X inside each box in the boxplots represents the mean and horizontal line the median value of each data group. “**” and “*” indicate significant difference from control at p<0.01 and 0.05 levels, respectively

FIG. 6A shows phenotypic difference of transgenic sorghum (42-1, 42-2) vs plant height. X inside each box in the boxplots represents the mean and horizontal line the median value of each data group. “**” and “*” indicate significant difference from control at p<0.01 and 0.05 levels, respectively.

FIG. 6B shows phenotypic difference of transgenic sorghum (42-1, 42-2) vs biomass (B), juice yield (C), sugar yield (D), tiller number (E), and thousand seed weight (F). X inside each box in the boxplots represents the mean and horizontal line the median value of each data group. “**” and “*” indicate significant difference from control at p<0.01 and 0.05 levels, respectively.

FIG. 6C shows phenotypic difference of transgenic sorghum (42-1, 42-2) vs juice yield. X inside each box in the boxplots represents the mean and horizontal line the median value of each data group. “**” and “*” indicate significant difference from control at p<0.01 and 0.05 levels, respectively.

FIG. 6D shows phenotypic difference of transgenic sorghum (42-1, 42-2) vs sugar yield. X inside each box in the boxplots represents the mean and horizontal line the median value of each data group. “**” and “*” indicate significant difference from control at p<0.01 and 0.05 levels, respectively.

FIG. 6E shows phenotypic difference of transgenic sorghum (42-1, 42-2) vs tiller number. X inside each box in the boxplots represents the mean and horizontal line the median value of each data group. “**” and “*” indicate significant difference from control at p<0.01 and 0.05 levels, respectively.

FIG. 6F shows phenotypic difference of transgenic sorghum (42-1, 42-2) vs thousand seed weight. X inside each box in the boxplots represents the mean and horizontal line the median value of each data group. “**” and “*” indicate significant difference from control at p<0.01 and 0.05 levels, respectively.

FIG. 7A shows phenotypic difference of transgenic sugarcane (Ts-1) in plant height. X inside each box in the boxplots represents the mean and horizontal line the median value of each data group. “**” and “*” indicate significant difference from control at p<0.01 and 0.05 levels, respectively.

FIG. 7B shows phenotypic difference of transgenic sugarcane (Ts-1) in biomass (B). X inside each box in the boxplots represents the mean and horizontal line the median value of each data group. “**” and “*” indicate significant difference from control at p<0.01 and 0.05 levels, respectively.

FIG. 8A shows phenotypic difference of transgenic sugarcane (Ts-1) in Plant height. X inside each box in the boxplots represents the mean and horizontal line the median value of each data group. “**” and “*” indicate significant difference from control at p<0.01 and 0.05 levels, respectively.

FIG. 8B shows phenotypic difference of transgenic sugarcane (Ts-1) in biomass. X inside each box in the boxplots represents the mean and horizontal line the median value of each data group. “**” and “*” indicate significant difference from control at p<0.01 and 0.05 levels, respectively.

FIG. 8C shows phenotypic difference of transgenic sugarcane (Ts-1) in Brix. X inside each box in the boxplots represents the mean and horizontal line the median value of each data group. “**” and “*” indicate significant difference from control at p<0.01 and 0.05 levels, respectively.

### BACKGROUND

Sugar is critical to world food supply because, except for rice, sugar prices influence all agricultural commodity prices. Thus, there is consistent interest to improve sugar yield in sugar crops, such as sugarcane, which accounts for 80% of sugar produced worldwide. However, scientific progress in genetic improvements of sugar yield in sugarcane has been slow.

The average sugarcane sugar yield in the US was flat from 1980-2010. In contrast, during that same time period, corn yield increased 68%. In Brazil, the world's leading sugarcane producer, sugar yield increased by a meager 34% in 35 years from 1975-2010 due to low genetic diversity and sugarcane's polyploidy genome.

Among cultivated grasses, sugarcane is most closely related to sorghum, a diploid. Both sugarcane and sorghum are C4 plants capable of accumulating large amounts of sucrose in the mature internodal stems. Although traits related to sucrose yield have been mapped in sorghum and sugarcane, candidate genes regulating sucrose accumulation and yield have not been identified in sorghum or sugarcane. This invention is the use of a pleiotropic sorghum gene that increases biomass and sugar yield in both sorghum and sugarcane.

SbSNF4 increases plant biomass, height, stalk extractable juice weight, juice sugar content, and seed weight when overexpressed. Biomass, height, juice weight, Brix, seed weight, tiller number are mapped to a single genetic locus on sorghum chromosome 6 close to SbSNF4 (Sb06g014920/Sobic.006G061100). Overexpression of SbSNF4 almost perfectly replicated the mapped phenotypic traits. Although homologs of SNF4 are not well studied in the prior art, its heterotrimeric binding partner, SnRK1, produced similar phenotypes when overexpressed in various plants from various sources. SNF4 genes will be important in sugar production as well as in enhancing other economically important traits in plants. Its use in this aspect can be through production of GMO plants or production of non-GMO plants via molecular breeding.

### DETAILED DESCRIPTION

The subject matter of the present invention is described with specificity herein to meet statutory requirements. However, the description itself is not intended to necessarily limit the scope of claims. Rather, the claimed subject matter might be embodied in other ways to include different steps or combinations of steps similar to the ones described in this document, in conjunction with other present or future technologies.

The 242 accessions of the sorghum mini core (MC) collection and 304 accessions of the Reference Set were phenotyped in rainy (denoted as 2010R, 2011R, and 2012R etc.) and post-rainy seasons with irrigation (denoted as 2010PRi and 2011PRi) and without irrigation (denoted as 2010PR and 2011PR). The plants were grown in an alpha design with three replications denoted as 1, 2, and 3 following the environment designation. Each single-row plot was 4 m long with a row spacing of 75 cm, and plant spacing within a row of 10 cm. Ammonium phosphate was applied at the rate of 150 kg/ha before planting, and 100 kg/ha of urea was applied as top dressing 3 weeks after planting. For post-rainy season with irrigation, field plots were irrigated five times at equal intervals each with 7 cm water. For MC, two weeks after anthesis, five representative plants were weighed to measure fresh biomass yield. After weighing, juice from each batch of five plants was extracted and Brix, juice volume and weight were measured; Brix was measured with a hand-held refractometer. Sugar yield can be calculated by multiplying juice weight with Brix. For both MC and RS, plant height, flowering time (days to 50% flowering), number of basal tillers, flag leaf chlorophyll content before (SPAD I) and after anthesis (SPAD II) were also recorded. SPAD was determined using a SPAD 502 (Minolta Spectrum Technologies Inc., Plainfield, Ill., USA) portable leaf chlorophyll meter.

The 265,500 SNP markers developed by Morris et al. (2013) were used in the association analysis. All SNPs start with “S” followed by chromosome number and physical position in the chromosome in base pairs (bp). These SNP markers have been validated for association analysis through high resolution mapping of the four sorghum brachytic height genes using another association panel. For association mapping in this study, the mixed linear model was used as implemented in TASSEL 5.0. Previous studies in maize, barley, and sorghum have shown that MLM with kinship index (K model) produces similar results compared with MLM with K and population structure indices (QK model) or MLM with K and principal component indices (PK model). The test with a smaller number of SNPs also supports the conclusion (data not shown). Therefore, only the K model was used and the kinship index was generated with SNP markers developed in a previous study. Associations between marker and trait were declared if multiple SNPs were linked with the trait in the same locus with p<0.0001 in more than one environment. Candidate genes containing linked SNP markers were identified based on information in Sbi1.4 on http://www.plantgdb.org/SbGDB/.

Vector and construct preparation. To make pUBI1301, pCAMBIA1301 was digested with PstI/EcoRI to remove the PstI/EcoRI fragment. This still left the HindIII site intact. The linearized pCAMBIA1301 without the PstI/EcoRI fragment was blunt-ended and self-ligated. The new vector was digested with HindIII and ligated with the HindIII fragment from pAHC25 containing the maize ubiquitin 1 (UBI) promoter and the GUS gene which can be replaced by a transgene through XmaI/SacI (Sm/Sc) digestion. The coding sequences of candidate genes were synthesized by Bio Basic Inc. (Amherst, N.Y.) or Synbio Technologies (Monmouth Junction, N.J.) and were delivered in pUC57 flanked by XmaI and SacI. Both synthesized gene and pUBI1301 were digested with XmaI and SacI to produce transgene constructs used in sorghum and sugarcane transformation.

Agrobacterium preparation. Competent Agrobacterium tumefaciens strain LBA4404 cells were transformed with the above transgene constructs using electroporation. A single colony from transformed Agrobacterium cells was inoculated into 10 ml LB broth with 50 mg l⁻¹ kanamycin and grown for 48 hours (h) at room temperature (RT). An aliquot of the cultured cells were subsequently inoculated into 200 ml LB with 50 mg l−1 kanamycin and grown for another 48 h. This culture was harvested for sugarcane transformation described below.

Sorghum genetic transformation. The procedure described by Liu and Godwin (2012) was followed. Immature panicles were collected 12-15 days after pollination. Seeds were removed from the panicles and soaked in 70% ethanol (v/v) for 5 min while shaking at 200 rpm. The soaked seeds were then drained and transferred to 50% commercial bleach shaken for 10 min before washed five times with sterilized water. Immature embryos ranging from 1.0 to 2.0 mm in length were isolated onto petri dishes containing callus induction medium (CIM: MS medium supplemented with 1 g/L L-proline, 1 g/L L-asparagine, 1 g/L KH₂PO₄, 0.16 mg/L CuSO₄ and 1 mg/L 2,4-D) with scutellums facing upward. The embryos were incubated in the dark in a tissue culture room before microprojectile transformation. Six embryos were placed at the center of a shallow petri dish containing osmotic medium (MS medium supplemented with 0.2 M D-sorbitol and 0.2 M D-mannitol) and stored for 2-3 h in the dark prior to bombardment which was performed using biolistic PDS 1000/He (Bio-Rad). Plasmid DNA delivery occurred via 0.6 μm gold particles (0.42 mg per shot). The distance from the filter holder to the target cells was adjusted to 12 cm and rupture disks is 1,100 P I. As much as 5 μg of each pNPTII (UBI: :NPTII) plasmid and each of the above transgene construct plasmids were mixed and then equally loaded into the receptacle for six shots. After bombardment, immature embryos were kept on osmotic medium for 3-4 h before being transferred onto CIM. After immature embryos recovered on CIM for 3-4 days, they were transferred to selective regeneration medium (MS medium supplemented with 1 mg/L BAP, 1 mg/L IAA, 0.16 mg/L CuSO4, 30 mg/L geneticin G418) and placed under lights in a tissue culture room. Immature embryos were subcultured every two weeks until putative transgenic shoots grew to 4-6 cm. These shoots were then moved to selective rooting medium (MS medium supplemented with 1 mg/L NAA, 1 mg/L IAA, 1 mg/L IBA and 0.16 mg/L CuSO₄, 30 mg/L geneticin G418) for 4 weeks without subculture. Rooted plantlets were transferred into plastic pots in greenhouse and were transferred into the field after 7 days in plastic pots.

Sugarcane genetic transformation. The procedures described by Mayavan et al. (2015) were used. Sugarcane cultivars Ho 02-113 and Co. 290 were provided by Jeffrey W. Hoy and Kenneth Gravois of Louisiana State University and L 01-299 by Garrie Landry. Sugarcane setts approximately 7 cm long were incubated in 1% carbendazim solution for 1 h and then rinsed several times with sterile water. Agrobacterium culture from above were pelleted and resuspended in infiltration medium (MS, 5% sucrose, 0.1% silwett L-77, 100 μM acetosyringone (AS) with an OD600 of 0.6. The axillary bud was gently pricked 5 times in 1 mm depth using a sterile 22 gauge hypodermic needle. The pricked setts were vacuum infiltered for 5 min at 500 mmHg in the suspension solution and were incubated in the suspension for 5 h at RT. The setts were then removed, air dried briefly on sterile paper towels and incubated (co-cultivated) at RT for 18 h in a desiccator under complete darkness. After co-cultivation, the setts were washed with sterile double-distilled water containing 500 mg/L cefotaxime to kill residual Agrobacterium tumefaciens before transferred to tissue culture boxes and partially immersed in 100 ml of sterile distilled water with 20 mg/L hygromycin and 500 mg/L cefotaxime. This antibiotic water was replaced weekly to avoid the bacterial growth. After about 30 days, putative transgenic shoots grew and planted in pots in greenhouse.

Transgenic sorghum plants were grown in Sanya, Hainan and Fengyang, Anhui, China. Transgenic sugarcane setts were grown in Lafayette, La., USA. For transgenic sorghum, Brix was measured by a hand-held refractometer either six weeks after anthesis or at harvest. To measure Brix, plants were harvested and fresh biomass was weighed before stripped off leaves and pressed for juice. Juice weight and volume were recorded and Brix was then measured. In addition, plant height, days to 50% flowering and number of basal tillers were also recorded. For transgenic sugarcane, all tillers from each sett were tested by PCR for the presence of hygromycin gene using the primers 5′-GATGTTGGCGACCTCGTATT-3′ and 5′-GATGTAGGAGGGCGTGGATA-3′. Canes were harvested after growing for 10 months (first six months in greenhouse and last four months outside). Plant height and fresh weight were recorded. To measure juice weight, internode of ˜5 cm in length was weighed and pressed with a hand-held cane juice presser. The resultant juice was weighed and Brix was measured with again a hand-held refractometer. The number of tillers was also recorded. T-test between transgenic plants and control was performed in https://www.graphpad.com/quickcalcs/ttest2/.

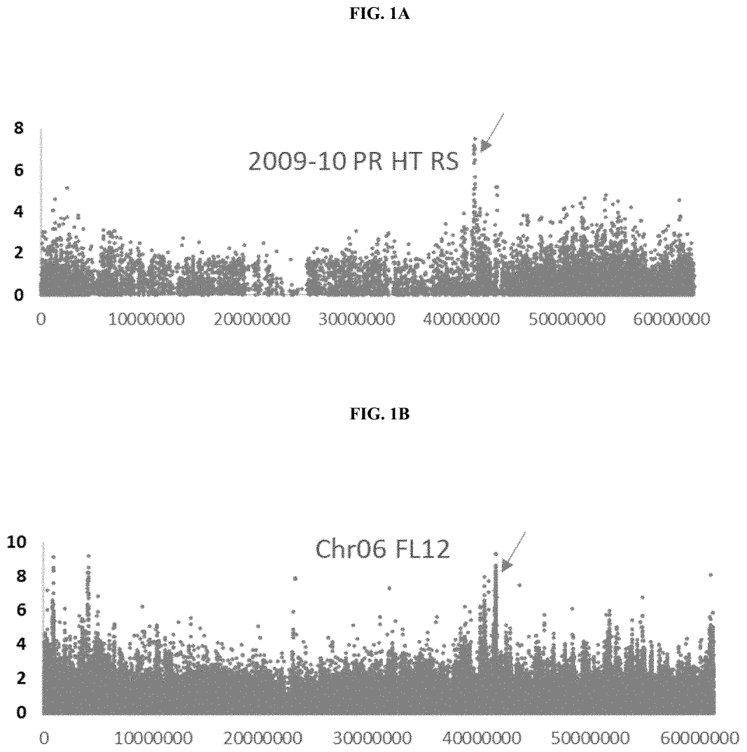

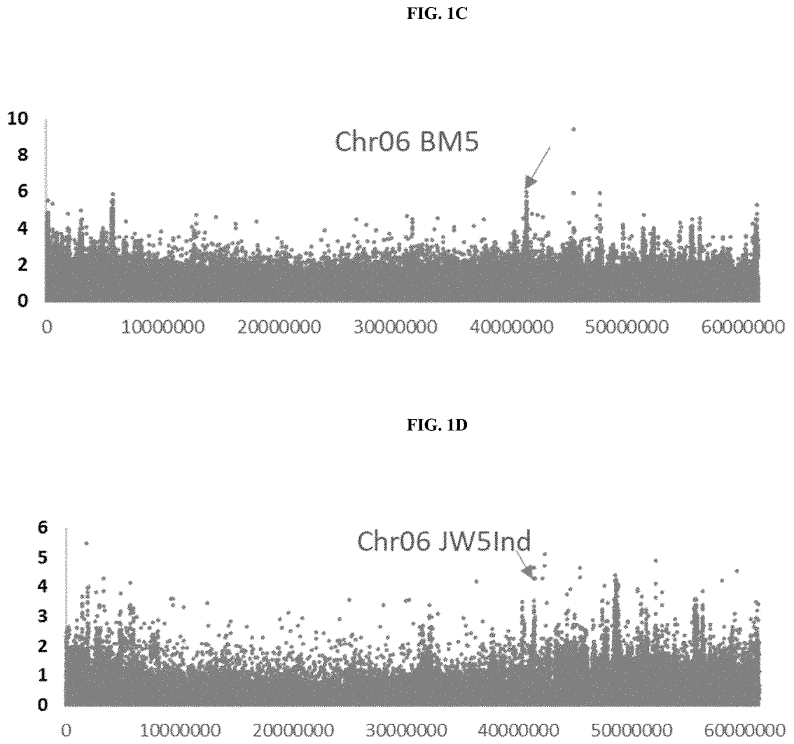

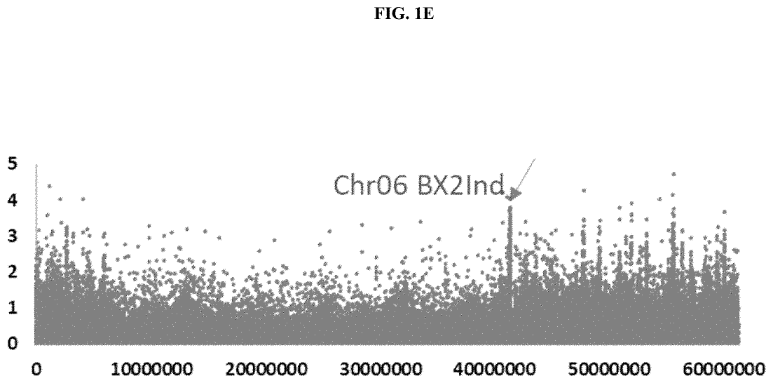

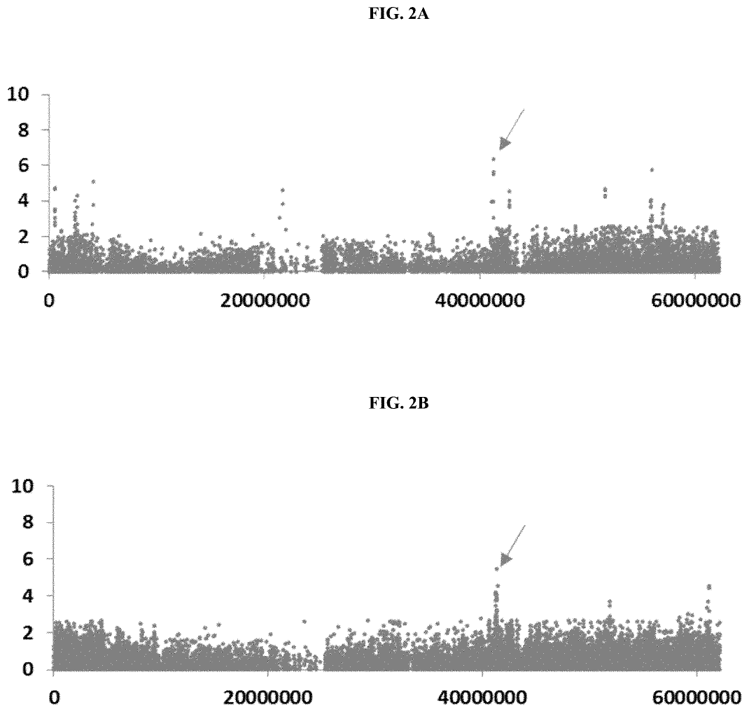

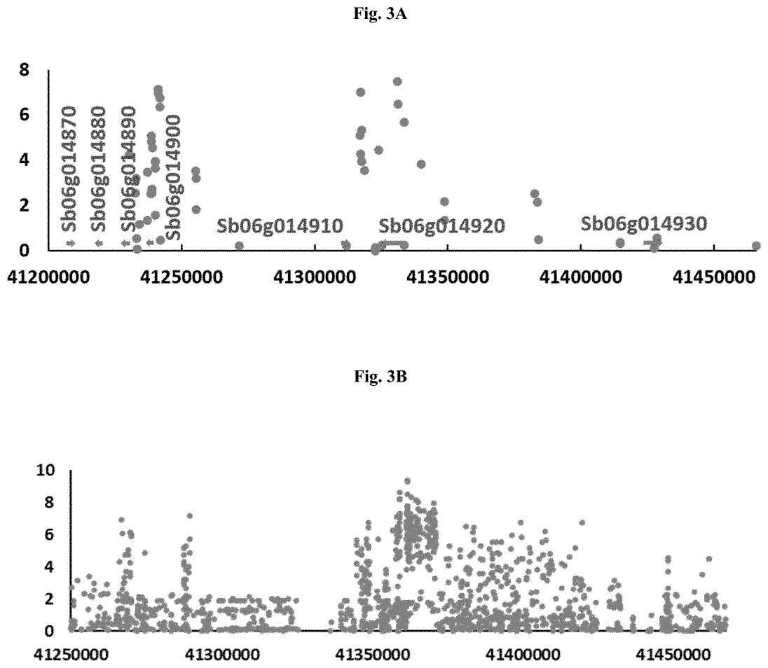

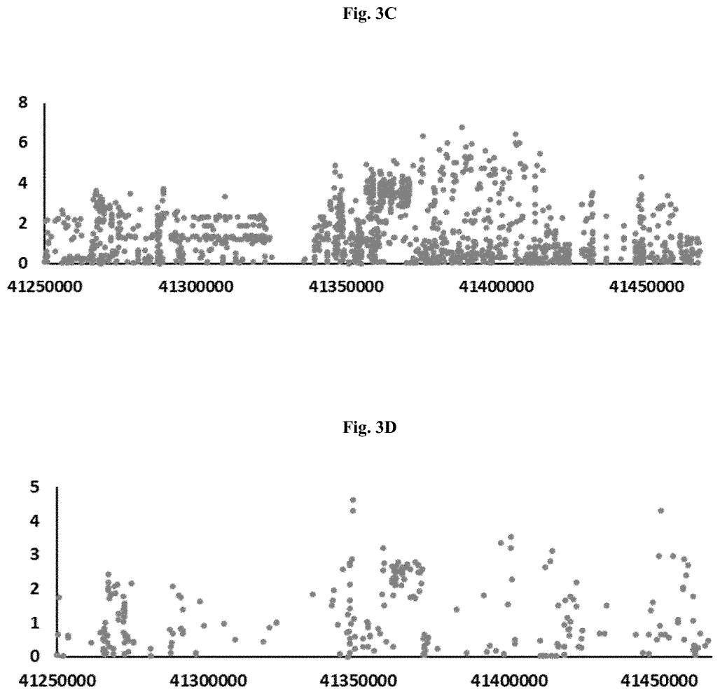

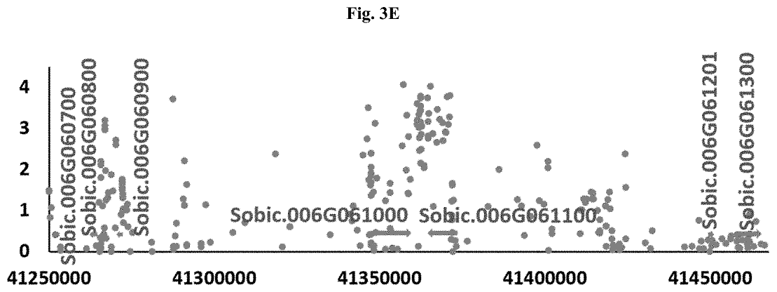

Two association mapping panels were used: MC and RS. Plant height and flowering time were phenotyped in both panels. Association mapping of phenotypes in MC and RS in seven environments identified a pleiotropic locus linked to biomass, Brix, juice weight, and flowering time in MC and flowering time and height in RS (FIG. 1A-1E). The peak in FIG. 1A-1E representing the pleiotropic locus is located on sorghum chromosome 6. In MC, the peak was observed in 2010R, 2011R and 2012R for biomass, 2011PRi and 2011PR for Brix, 2011R and 2012R for juice weight, all nine testing environments for flowering time (DFF: days to 50% flowering). In RS, the peak was observed in 2008PRi and 2008PR for DFF, and in 2008PRi, 2008PR, 2009PRi and 2009PR for plant height (HT). Manhattan plot covering 41150 kb-41350 kb of these peaks are presented in FIG. 3A-3E. Association of the locus with tiller number or 100 seed weight was not strong in any environment and only observable with averaged phenotypic values (FIG. 2A, 2B).

Based on the sorghum genome Sbi1.4, there are seven genes covered/flanked by the locus as shown in Table 1. Genes 3 and 4 are homologs of the sugarcane Scdr1. Gene 5 shares similarity to Scdr1 and extension and is also annotated as PYRICULARIA ORYZAE RESISTANCE 21 (XP_021319810) but is not expressed in seeds according to expression data on Phytozome. Therefore, Genes 4, 6 and 7 were used for genetic transformation experiments.

TABLE 1
Candidate genes in the pleiotropic
locus on sorghum chromosome 6.

| Gene # | Gene ID | Start-Stop (Sbi1.4) | Annotation |
|---|---|---|---|
| 1 | Sb06g014865 | 41186630-41187007 | extensin |
| 2 | Sb06g014870 | 41208376-41210142 | Predicted protein |
| 3 | Sb06g014880 | 41219449-41221163 | Scdr1 (sugarcane |
| | | | drought- |
| | | | responsive 1) |
| 4 | Sb06g014890 | 41230021-41231850 | Scdr1 |
| 5 | Sb06g014900 | 41238494-41239811 | extensin/similar |
| | | | to Scdr1 |
| 6 | Sb06g014910 | 41315548-41316972 | pentatricopeptide |
| | | | repeat-containing |
| | | | (Slo1) |
| 7 | Sb06g014920 | 41330675-41338760 | sucrose |
| | | | nonfermenting |
| | | | 4-like protein |
| | | | (SbSNF4) |

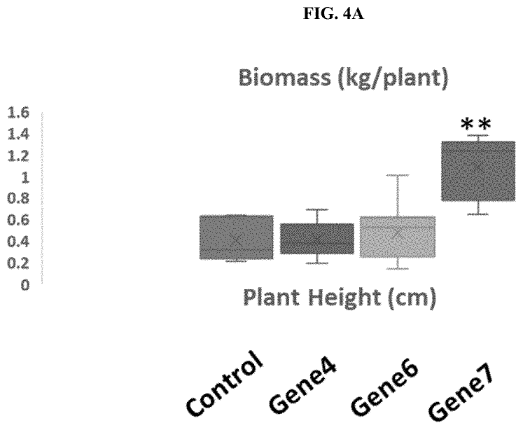

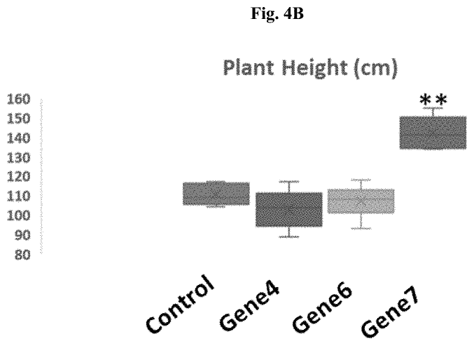

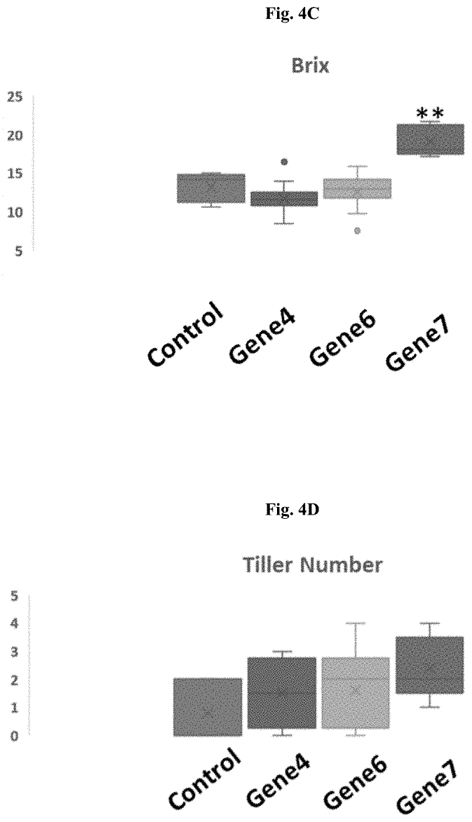

Sorghum immature embryos from Tx430 were transformed with overexpression constructs of Genes 4, 6, and 7 listed in Table 1 and FIG. 3 using particle bombardment. Five transformants each were generated for Genes 4 and 6, but only one was generated initially for Gene 7. T1 generation of these transformants were evaluated for biomass, plant height, tiller number and Brix. Results from this phenotypic evaluation are presented in FIG. 4A, FIG. 4B, FIG. 4C, and FIG. 4D. They show that only Gene 7 transgenic plants displayed significantly higher phenotypic values in biomass, plant height, and Brix. Although tiller number in the Gene 7 transgenic plants was higher, it is not statistically significant. Based on these results, Gene 7 (Sb06g014920/Sobic.006G061100/SbSNF4) was determined to be the gene underlying pleiotropic phenotypes. Further transformation produced another Gene 7 overexpression plant hereafter named 7-2 and the Gene 7 overexpression line shown in FIGS. 4A-4D is named 7-1. T1 and T2 generations of both overexpression lines were further analyzed and are presented in the following section.

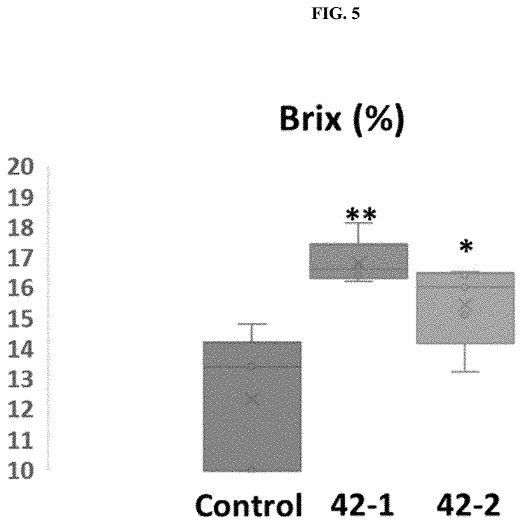

As shown in FIG. 4C, Brix in 7-1 increased by 44% (30-63%) on average over control, significant at p<0.01 level. In a separate field evaluation, this increase was more than 185% on average (FIG. 5). In 7-2, the average increase in Brix over control was 38% (14-87%), significant at p<0.05 level (FIG. 5).

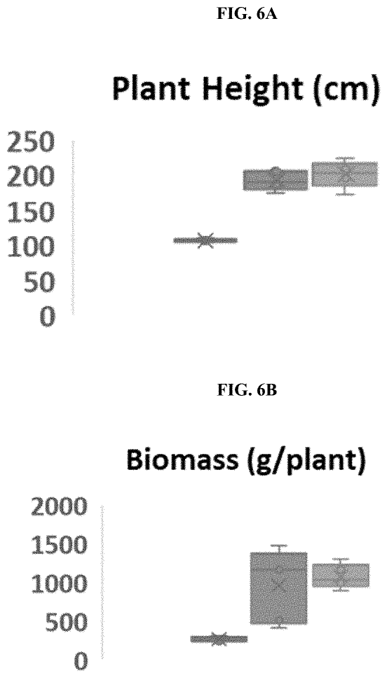

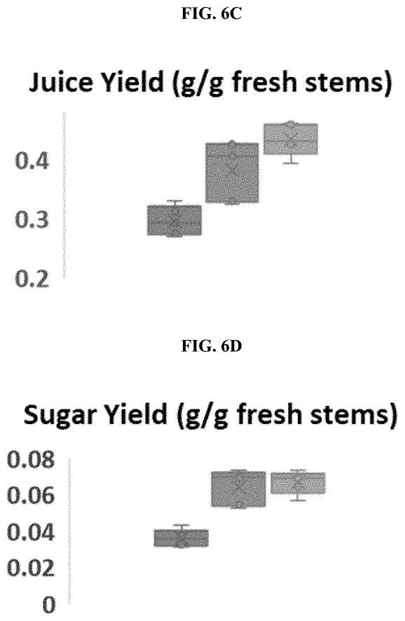

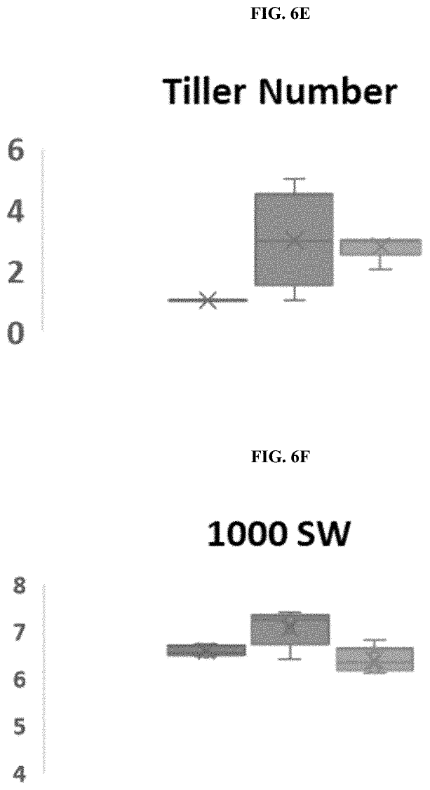

In addition to Brix, both 7-1 and 7-2 SbSNF4 overexpression lines also displayed increased biomass, height, juice weight, tiller number and 100 seed weight compared to control (FIG. 6). Furthermore, in May 3, 2019 planting, 7-1 flowering date was delayed by three days and that of 7-2 by one. It has been consistently shown thus far that 7-1 produced more dramatic phenotypes than 7-2 plants. The fact that all phenotypic changes also match those traits mapped by association mapping provides further evidence that SbSNF4 is the gene of the pleiotropic effect on sorghum chromosome 6.

Inoculation of three sugarcane varieties, Ho 02-113, Co. 290 and L 01-299, with 300 setts each only produced one putative transgenic sett (OE-B) from Ho 02-113. The sugarcane sett was initially grown in greenhouse and later transplanted outside. OE-B was tested by PCR for the presence of hygromycin sequence. PCR results indicated the presence of the hygromycin gene in all tillers from the sett.

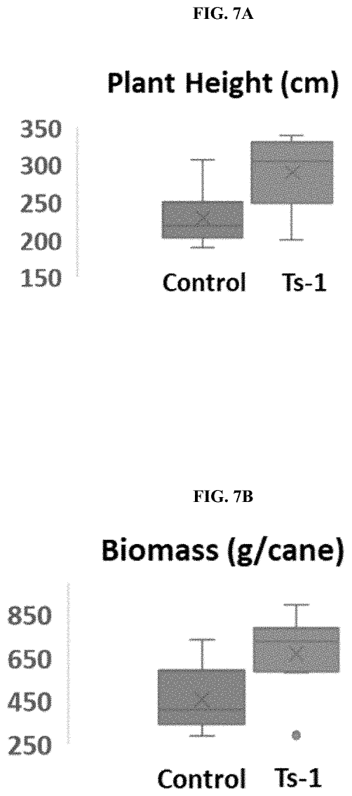

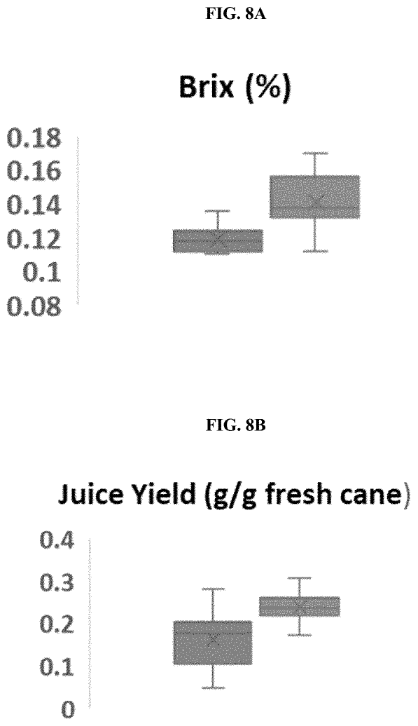

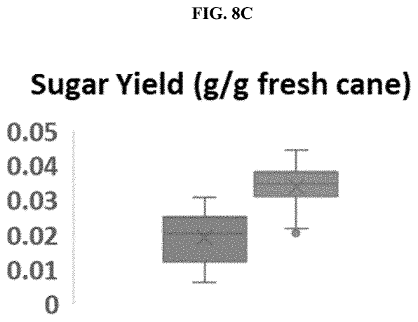

The OE-B plant was on average 13% taller than control (FIG. 7A). They also produced more juice per cane (FIG. 7B). On per unit of cane weight and on average, OE-B plant (FIG. 8A) produced 93% more juice than control. For Brix, OE-B was 27% higher than control (FIG. 8B).

The evolutionarily conserved AMPK/SNF1/SnRK1 (sucrose non-fermenting1-related protein kinases 1) kinase heterotrimeric complexes typically consist of a catalytic α, a scaffolding β and an activating γ subunits and play central regulatory functions in metabolism, stress signaling and development. In Arabidopsis, AKIN10 (At3g01090) and AKIN11 (At3g29160), the α subunits of the SnRK1 complex, were found to regulate the expression of more than 600 target genes in response to starvation or nutrient signals in protoplasts. To support their roles in growth and development, both AKIN10 (At3g01090) and AtSNF4 (AT1G09020) are highly expressed in shoot meristems, elongating and differentiating zones of primary and lateral roots, as well as in young leaf primordia.

In yeast and mammals, the γ subunit acts as the complex's energy-sensing module by controlling the activity of the α subunit. The γ (or βγ in plants) subunits typically comprise four highly conserved cystathionine-β synthase (CBS) motifs that can bind adenine nucleotides and hence function as the cellular energy-sensing module of the complex. In Arabidopsis, βγ (AtSNF4; AT1G09020) is the ortholog of the yeast SNF4, the yeast γ subunit gene. Emanuelle et al. (2015) demonstrated that βγ/AtSNF4 is essential for SnRK1 heterotrimeric formation—in Arabidopsis, six heterotrimeric SnRK1 isoenzymes exist, each containing one of two α-subunits, one of three β-subunits, and, in all cases, the one βγ-subunit. The sorghum SNF4 presented here, SbSNF4, is most homologous to AtSNF4 (AT1G09020) in Arabidopsis based on BLAST analysis.

Very few studies on βγ/SNF4 subunits in plants can be found in the prior art. However, many studies on SNF1/SnRK1 overexpression have been published. Interestingly, many such studies produced similar phenotypes presented here. For example, overexpression of AKIN10/SnRK1.1 (At3g01090) and maize SnRK1s (ZmSnRK1.1, ZmSnRK1.2 or ZmSnRK1.3) delays flowering time in Arabidopsis; overexpressing ZmSnRK1.1, ZmSnRK1.2 or ZmSnRK1.3 in Arabidopsis also increases plant height, biomass and seed weight. AKIN10 also regulates lateral organ development. In sugarcane, the AKIN10 homolog scjfrz2032g01.g has high expression in high-Brix and low expression in low-Brix varieties. Similarly, overexpression of AKIN10/SnRK1.1 in Arabidopsis and potato StSnRK1 in tobacco increases leaf glucose and fructose content by 30% in Arabidopsis and leaf sucrose and soluble sugar by 34-82% and 42-54%, respectively, in tobacco. In storage organs, overexpression of crabapple MhSnRK1 and peach PpSnRK1α in tomato increases fruit soluble sugar by over 30% in both cases.

For the purpose of understanding A Pleiotropic Gene that Increases Biomass and Sugar Yield in Sorghum and Sugarcane, references are made in the text to exemplary embodiments of A Pleiotropic Gene that Increases Biomass and Sugar Yield in Sorghum and Sugarcane, only some of which are described herein. It should be understood that no limitations on the scope of the invention are intended by describing these exemplary embodiments. One of ordinary skill in the art will readily appreciate that alternate but functionally equivalent components, materials, designs, and equipment may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

1. A method for increasing biomass and sugar yield of a plant comprising:
a. transforming a plant with a first gene that is functional in a plant,
wherein said gene is pleitropic, and
wherein said plant overexpresses said gene, thereby increasing biomass and sugar yield;
b. producing said plant through molecular breeding using information of said gene; and
c. cultivating said plant.

2. The method of claim 1, wherein said gene is Sb06g014920/Sobic.006G061100/SbSNF4.

3. The method of claim 1 wherein said plant is of the species saccharum officinarum.

4. The method of claim 1 wherein said plant is of the genus sorghum.

5. A genetically modified plant comprising overexpression of a pleitropic gene that increases biomass and sugar yield.

6. The genetically modified plant of claim 5 wherein said plant is sorghum.

7. The genetically modified plant of claim 5 wherein said plant is sugarcane.

8. The genetically modified plant of claim 5 wherein said pleitropic gene is Sb06g014920/Sobic.006G061100/SbSNF4.

9. A method of producing a transgenic plant having enhanced plant biomass and sugar yield comprising introducing into a plant Sb06g014920/Sobic.006G061100/SbSNF4 such that Sb06g014920/Sobic.006G061100/SbSNF4 becomes overexpressed.

10. The method of claim 9 wherein said introducing is performed by transforming immature plant embryos with overexpression constructs of Sb06g014920/Sobic.006G061100/SbSNF4 using particle bombardment.

11. The method of claim 9 wherein said transgenic plant is selected from the group consisting of: sugar cane and sorghum.
